(19) United States
(12) Patent Application Publication
(10) Pub. No.: US 2008/0119893 A1
(43) Pub. Date: May 22, 2008

(54) Denatured human albumin suture material

(76) Inventor: Yasmin Wadia, Humble, TX (US)

Correspondence Address: ELIZABETH R. HALL, 1722 MARYLAND STREET, HOUSTON, TX, 77006, US

(21) Appl. No.: 11/973,833

(22) Filed: Oct. 10, 2007

Related U.S. Application Data
(60) Provisional application No. 60/866,571, filed on Nov. 20, 2006.

(51) Int. Cl. A61L 17/06; A61B 17/04

(52) U.S. Cl. 606/230

(57) ABSTRACT

The present invention provides a biocompatible suture material made from denatured human serum albumin. The suture material can be constructed by molding and denaturing a liquid human serum albumin, or by precision cutting a denatured albumin lamina into thin, threadlike ribbons. The denatured albumin material is strong, flexible, absorbable and biocompatible based on both in vitro and in vivo testing.

### CROSS-REFERENCE TO RELATED APPLICATION

The present application, pursuant to 35 U.S.C. 111(b), claims the benefit of the earlier filing date of provisional application Ser. No. 60/866,571 filed Nov. 20, 2006, and entitled “Denatured Human Albumin Biomaterial as Burns, Wound Dressing and Sutures, Applications and Manufacture.”

### BACKGROUND OF THE INVENTION

1. Field of the Invention

The invention relates to a biocompatible suture material formed from a denatured human serum albumin. More particularly, the present invention relates to a suture material made by denaturing a liquid solution containing at least 47% human serum albumin.

2. Description of the Related Art

Suture materials are one of the ways that physicians use to repair and close wounds. Other methods or materials used for wound closure and repair include surgical staples, surgical adhesive strips, and surgical glues. Each type of wound closure and repair material has its advantages and disadvantages, although suture materials are still one of the most commonly employed. A wide variety of suture materials is currently commercially available for use both internally and externally for wound repair and wound closure. Improved designs of suture materials are sought in order to provide for faster wound healing with less chance of infection and the least amount of local inflammation, injury and scar formation. The present invention is a biocompatible suture material prepared from denatured human albumin.

Denatured human serum albumin has been recognized as a safe and effective biomaterial with a variety of applications. It has been employed as a food additive and fat replacement agent (U.S. Pat. No. 7,166,316), as a material for construction of drug delivery devices (U.S. Pat. No. 4,666,641), and as a component of implantable materials in order to inhibit thrombogenesis (U.S. Pat. No. 5,632,776). These applications of denatured human serum albumin demonstrate the versatility of the substance as well as the biocompatibility of the basic product. The U.S. Food and Drug Administration has approved human serum albumin as safe and effective in some of these applications.

U.S. Pat. No. 6,680,063 B1 discloses a denatured human serum albumin composition and methods for making the composition, as well as application of the denatured albumin compositions for repair of tissue defects and lesions. The denatured human serum albumin product of this patent is formed into sheets that can be then molded or cut by a surgeon as needed during surgery for tissue repair. The denatured albumin compositions contained from 50% to 58% albumin and comprises a thin, pliable sheet formed to a thickness of from 75 μm to about 300 μm. The patent also discloses applying the biocompatible denatured albumin material to a solid visceral organ along with an energy-absorbing proteinaceous material and irradiating the materials in order to fuse the denatured albumin and the proteinaceous material to the tissue for tissue repair. The patent discloses that the denatured human albumin sheets have the strength for tissue repair and the biocompatibility required for internal organ exposure, while allowing for efficient wound healing.

There is a long standing need for a truly biocompatible suture material that is also bioabsorbable.

### SUMMARY OF THE INVENTION

The present invention is a biocompatible surgical suture material containing a denatured human serum albumin. In one embodiment, the suture material is made by denaturing a solution of human serum albumin. The solution contains a concentration of from 47% to 58% human serum albumin, or a preferred concentration of about 50% to 54%.

In one embodiment, a surgical suture is formed by molding liquid human serum albumin solution in a tubular mold or dye and denaturing the extruded liquid albumin by application of wet or dry heat at a temperature of from 85° C. to about 120° C. for from 15 seconds to about 30 minutes. In another embodiment, the biocompatible surgical suture is formed by cutting fine ribbons from denatured human serum albumin sheets. Also provided are methods for wound repair and closure by suturing with the biocompatible surgical sutures of the present invention.

A further embodiment of the present invention is a surgical suture consisting essentially of a denatured human serum albumin.

Another embodiment of the present invention is a surgical suture containing at least 50% w/v of a denatured human serum albumin, wherein the suture has a yield strength ranging from about 800 kilopascals to about 1200 kilopascals and a Young's modulus of elasticity ranging from about 2500 kilopascals to about 3500 kilopascals.

Yet another embodiment of the present invention is a method for making a suture material comprising the steps of: obtaining an albumin solution having a concentration of human serum albumin ranging from about 47% w/v to about 58% w/v; casting the albumin solution into a predetermined shape; and denaturing the human serum albumin by heating the albumin to at least 85° C. at a pressure of at least 1 atmosphere for at least 15 seconds.

Yet another embodiment of the present invention is a method for making a biocompatible suture material by molding an albumin solution containing 47% w/v to 58% w/v of human serum albumin solution in a tubular mold and heating the molded albumin solution at a temperature ranging from 85° C. to 120° C. for about 15 seconds to about 30 minutes at a pressure ranging from 1 atmosphere to 3 atmospheres.

The foregoing has outlined rather broadly several aspects of the present invention in order that the detailed description of the invention that follows may be better understood. Additional features and advantages of the invention will be described hereinafter which form the subject of the claims of the invention. It should be appreciated by those skilled in the art that the conception and the specific embodiment disclosed might be readily utilized as a basis for modifying or redesigning the structures for carrying out the same purposes as the invention. It should be realized by those skilled in the art that such equivalent constructions do not depart from the spirit and scope of the invention as set forth in the appended claims.

### BRIEF DESCRIPTION OF THE DRAWINGS

For a more complete understanding of the present invention, and the advantages thereof, reference is now made to the following descriptions taken in conjunction with the accompanying drawings, in which:

FIG. 1 depicts results of experiments to determine the yield strengths of albumin strips cured for various time periods at 100° C.

FIG. 2 depicts yield strengths for albumin strips that have been cured at 86° C. The thickness of the sheets is given on the vertical axis in micrometers (μm); for example, 270b means that the sample was the second sample with a thickness of 270 μm.

FIG. 3 depicts yield strengths of albumin strips cured at multiple temperatures, including albumin strips that were autoclaved.

FIG. 4 depicts the Young's modulus (kPa) for albumin strips that have been cured at 86° C. The thickness of the sheets is given on the vertical axis in μm; for example 270b means that the sample was the second sample with a thickness of 270 μm.

FIG. 5 depicts the Young's modulus (kPa) for albumin strips cured for various time periods at 100° C.

### DESCRIPTION OF THE PREFERRED EMBODIMENTS

The present invention relates to a novel biocompatible, bio-absorbable suture material made from denatured human serum albumin for use in wound repair and closure. The suture material is formed from denatured human serum albumin, hereafter referred to as denatured albumin. A “suture material” is herein defined as a strand of material used in securing the closure of a wound. “Biocompatible” is herein defined as a material that has low toxicity, low antigenicity, and minimal of immune rejection and foreign body reactions such as to meet FDA-approved standards for biocompatibility for mammalian, including human, use. “Biodegradable” or “bio-absorbable” are used interchangeably herein to refer to a material that is degraded and/or absorbed by the human body.

Human serum albumin is a composition that has been approved for human use by the U.S. Food and Drug Administration (FDA). FDA-approved human serum albumin has been established to be completely biocompatible and biodegradable. Unlike other biomaterials that are often derived from animal sources (e.g., collagen, elastin) human-derived biomaterials such as human serum albumin have less concern in terms of potential antigenicity, immune rejection and foreign body reactions. Additionally, human source materials avoid concerns attendant with animals such as transmission of animal diseases and viruses. Since the protein sequence and structure of serum albumin varies little among the human population, human serum albumin is a preferred biomaterial.

It has previously been shown that denatured albumin can be formed into a sheet or lamina structure (U.S. Pat. No. 6,680,063 B1). The denatured lamina is clear, flexible, thin, and can be prepared at a uniform thickness. Once prepared in sheets, the composition can be easily manipulated without special care due to its high tensile strength and pliability. Although the surface of the sheet has a slight tackiness, the material does not bond or stick to itself in a manner to interfere with its use in vivo.

Denaturation of the albumin results in a lamina that is stable in a variety of environments, including those that are characteristic of human physiology. The denatured lamina does not solubilize in water or saline solution. These properties allow the lamina to be repositioned and used after initial contact with tissue.

The denatured albumin lamina is stable for certain periods of time in air, remaining pliable for as long as 15 minutes when exposed to air. As a result, the denatured albumin product is stored under vacuum once manufactured, until use.

The denatured lamina is typically sterilized using an autoclave, gamma irradiation, or ethylene oxide gas. Because denaturation is desired, autoclaving the material can concurrently accomplish both the sterilization and denaturation steps in the manufacture of the lamina.

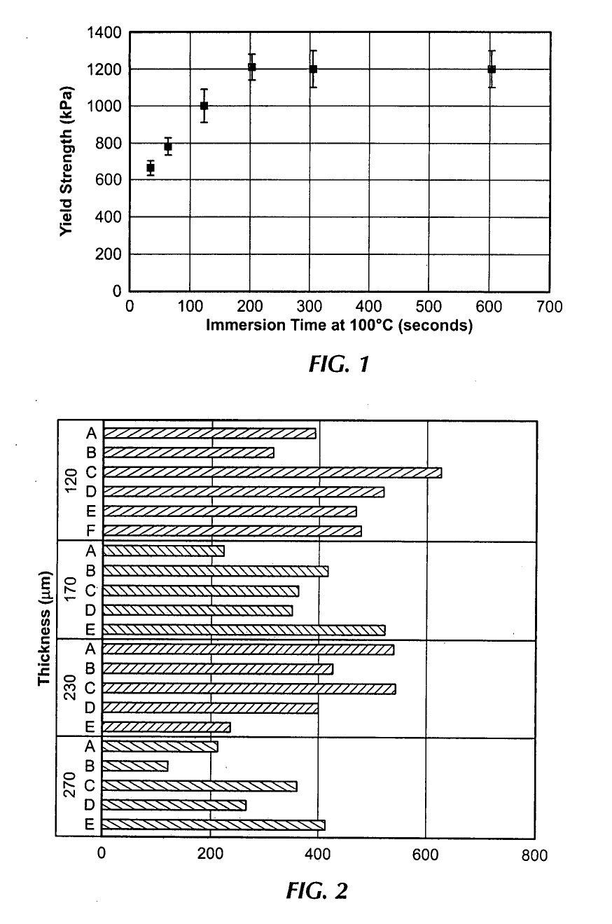

Studies have been performed to test the strength of the denatured albumin lamina. Albumin lamina was cured at 100° C. for 30, 60, 120, 200, 300, or 600 seconds in which a dog bone pattern die was used to cut albumin strips so that the failure point was consistently in the middle of the sample, rather than at the clamps. Strips of approximately 2×1 centimeter (cm) were stretched in a Chatillon Materials Tester. Ultimate strength, or yield strength, was calculated for the albumin strips by dividing the cross-sectional area of the test strip into the force required to break the test strip (FIG. 1). The results showed that yield strengths increased almost linearly with cure times from 30-200 seconds. Curing the albumin strips for longer than 200 seconds, however, did not significantly increase the yield strength of the material.

Ultimate strengths were also determined for albumin strips of different thicknesses cured at 86° C. for 30 seconds (FIG. 2). The albumin strips were cut from the albumin sheets with a dog bone pattern die so that the failure point was consistently in the middle of the sample, rather than at the clamps. The ultimate strengths were recorded along with the exact width and thickness of each sample. The ultimate strength was calculated by dividing the force required to break the sample by the cross-sectional area (width×thickness).

Multiple strips having a thickness of 120 μm, 170 μm, 230 μm, and 270 μm were tested. FIG. 2 shows the thickness of each strip on the vertical axis and the yield strength of each strip along the horizontal axis. The results for six denatured albumin strips having a thickness of 120 μm (labeled A-F), five albumin strips having a thickness of 170 μm (labeled A-E), five denatured albumin strips having a thickness of 230 μm (labeled A-E), and five denatured albumin strips having a thickness of 270 μm (labeled A-E) are shown in FIG. 2.

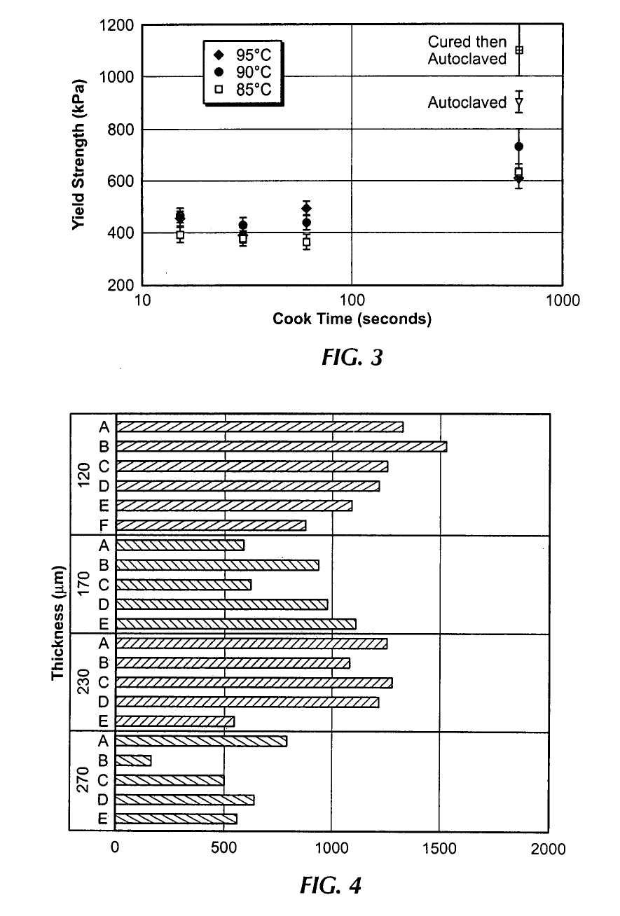

FIG. 3 illustrates the ultimate strengths in kilopascals (kPa) of albumin strips denatured by heat bath immersion at 85° C., 90° C., and 95° C. The ultimate strengths of two sets of autoclaved albumin strips were also measured for comparison. Once set of autoclaved materials was cured by autoclaving the material at 110° C. and the other set was initially cured by a 15-30 second heat bath immersion and then autoclaved at 110° C. The data illustrate a significant increase in the yield strength of the autoclaved denatured albumin strips and an insignificant increase in the yield strength of the denatured albumin strips cured at 85° C., 90° C., and 95° C. Furthermore, the results clearly illustrate that there was a significant increase in yield strength of the denatured albumin strips cured for 600 seconds versus the denatured albumin strips that were cured from 15 to 60 seconds.

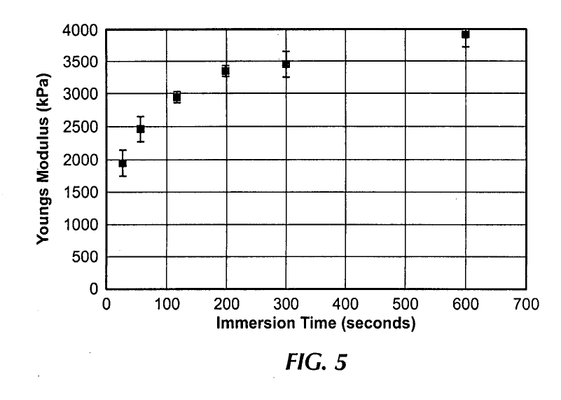

Experiments were also performed to test the elasticity of denatured albumin strips cured at 86° C. (FIG. 4) and 100° C. (FIG. 5). Young's modulus of elasticity was calculated for each sample by a linear fit of stress/strain data for strains ranging from 0 to 0.1.

The elasticity or stiffness of the denatured albumin strips of different thicknesses cured at 86° C. for 30 seconds was determined (FIG. 4). The Young's modulus of elasticity were recorded for multiple denatured albumin strips having a thickness of 120 μm, 170 μm, 230 μm, and 270 μm. FIG. 4 shows the thickness of each strip on the vertical axis and the Young's modulus (kPa) of each strip along the horizontal axis. The results for six denatured albumin strips having a thickness of 120 μm (labeled A-F), five denatured albumin strips having a thickness of 170 μm (labeled A-E), five denatured albumin strips having a thickness of 230 μm (labeled A-E), and five denatured albumin strips having a thickness of 270 μm (labeled A-E) are shown in FIG. 4.

Studies were performed to test the elasticity of denatured albumin strips cured for different time periods. Albumin strips were cured at 100° C. for 30, 60, 120, 200, 300, or 600 seconds. Young's modulus of elasticity was calculated for each sample by a linear fit of stress/strain data for strains ranging from 0 to 0.1. The results showed that the stiffness (Young's modulus) of the denatured albumin strips increased with increased curing time, with the majority of the effect seen within the first 200 seconds.

The data on strength and elasticity provided parameters for developing denatured albumin suture materials. The denatured albumin suture materials therefore were designed based on the mechanical properties of strength and elasticity. Preferred suture materials typically have a yield strength of at least 400 kPa (preferably ranging from about 800 kPa to about 1200 kPa) and an elasticity of less than 4000 kPa (preferably ranging from about 2500 kPa to about 3500 kPa).

Any method of manufacture that produces a thin, thread-like suture material that is strong, yet flexible, is contemplated by the present invention. However, two preferred methods of manufacture are described.

The first method involves preparation of denatured albumin sheets or lamina ranging from 50 μm to about 500 μm in thickness. The starting material for the lamina is a liquid human serum albumin solution of approximately 47% to 58% albumin concentration. As used hereinafter, the terms “percent” and “%” refer to weight per volume (gm/100 ml) unless otherwise noted.

The concentrated albumin solution is placed between two nonporous sheets (e.g., medical grade plastic or preferably PTFE or Teflon®). Typically the concentrated albumin solution is placed between the two aligned sheets. The sheets are rolled through graduated rollers to spread the albumin evenly and to a uniform thickness of from about 50 μm to about 500 μm. The rolled liquid albumin between the non porous sheets is subject to wet or dry heat ranging from about 85° C. to about 120° C. for 15-200 seconds which denatures the liquid albumin to form a solid.

Alternatively, sheets are cured at about 90° C. for about 15 seconds and then autoclaved at 110° C. for about 10 minutes. The liquid albumin may also be denatured by autoclaving alone from about 100° C. to about 135° C. The factors shown to influence the properties (e.g., yield strength and flexibility) of the resultant denatured lamina are the concentration of human serum albumin in the albumin solution (47% to 58%), curing temperature (85° to 135° C.), curing time (15 seconds to 30 minutes), and curing pressure (1 atm to 3 atm). The liquid albumin is preferably denatured at about 100° C. for about 120 seconds at a pressure of about 2 atmospheres. The concentration of the albumin solution, the curing temperature, curing time, and curing pressure are selected to optimize the characteristics of the denatured albumin suture material to meet the needs of a designated use.

The denatured sheets can then be precision cut into fine ribbons to create thin, thread-like suture material. The denatured albumin sheets are precision cut by lasers or roller blades in a graduated parallel arrangement spaced according to the gauge of the suture being produced (e.g., U.S.P 10/0 to 7 or having a diameter between 0.013 mm to 0.914 mm). The precision cutting is done at room temperature and pressure. Typically the length of the suture material produced is from about 10 inches to about 30 inches depending on the suture diameter, needle size to be used, and the application for use of the suture.

The second preferred method involves injection of liquid phase human serum albumin (47% to 58% w/v) into fine tubular molds or dyes which are then heated to between 85° C. to 120° C. wet or dry heat for about 15 seconds to as much as 30 minutes while being extruded in a continuous process. The molded product results in a fine, thread-like suture material.

The injection/extrusion process is similar to screw plastification (commonly used for processing plastics, rubber and ceramic materials) that includes extruding and injection molding the material being produced. When this process is used for plastics or rubber, the pre-extrusion temperature is typically >200° C. in order to liquefy solid polymer granules. As the liquefied polymer is extruded using a screw extruder, it is pushed through a mold or dye. Immediately post-extrusion, the polymer is actively cooled to obtain a solid shape.

In contrast to the other materials produced by this process, the preferred pre-extrusion temperature for 50-54% human serum albumin ranges from 4-37° C. and is preferably done at room temperature. The albumin solution is continuously extruded through a heated mold or dye using a graded extrusion pressure. Typically, a post-extrusion dry or wet heat (ranging from 85-120° C.) and pressure of 1-3 atmospheres is applied to the extruded albumin material to finalize the transformation of the liquid albumin solution into a denatured albumin solid.

Testing of the denatured albumin biomaterial has been performed to examine various properties of the material that would be critical to the application of the material for its use as surgical sutures, such as tensile strength, cytotoxicity and biocompatibility. Such testing is required by regulatory agencies during development of surgical sutures.

One such type of testing, cytotoxicity testing, is an important consideration for any material that is to be used as an implantable material, which would include surgical suture materials. In the present invention, cytotoxicity testing was performed on samples of denatured albumin in accordance with Good Laboratory Practice (GLP) regulations. The test performed, a MEM elution test, is a standard test procedure performed on all types of medical devices, including surgical sutures. Cytotoxicity testing is an in vitro test process that is a rapid and sensitive method to determine if the materials used contain significant quantities of harmful extractables, and then to quantify the effect of such extractables on cellular components.

In the MEM elution test, a test sample of extracted denatured albumin was placed in contact with a monolayer of mouse heteroploid connective tissue (L-929) cells and then incubated. The cells were then scored for cytotoxic effects (degree of cellular destruction).

More specifically, an extract of denatured albumin was prepared based on USP and ANSI/AAMI/ISO surface area recommendations or weight. The sample was extracted for 24 to 25 hours at 37° C. in 1× Minimal Essential Media with 5% calf serum. Positive (latex natural rubber) and negative (polypropylene pellets) controls were similarly extracted and included in the assay. A blank of extraction media (a “media control”) was also included in the assay. Multiple well cell culture plates were seeded with a verified quantity of L-929 cells and incubated until 80-90% confluent. The cell culture media was removed from the plates. The test extracts were filtered and the appropriate amount of extract was added to each well on the cell culture plates. Each extract was tested on three wells of cells. The cells were incubated at 37° C. with 5±1% CO² for 72±3 hours.

The cell monolayers were examined microscopically. The wells were scored as to the degree of discernable morphological cytotoxicity on a relative scale of 0 to 4 (0=no reactivity; 1=slight reactivity; 2=mild reactivity; 3=moderate reactivity; 4=severe reactivity). The results from the three wells were averaged to give a final cytotoxicity score.

The results showed that the denatured albumin extract samples exhibited mild activity only (score of grade 2 of 4) in the MEM elution test. Using the standards set forth by the United States Pharmacopeia (USP), the denatured albumin material is acceptable for human contact (i.e., a score no greater than 2).

In addition to the in vitro testing for cytotoxicity, in vivo tests of biocompatibility were also performed with the denatured albumin material. Two tests were performed, the murine local lymph node assay (LLNA) and the intracutaneous reactivity test. Both of these tests are standard tests of biocompatibility that are used in the development of materials for medical devices such as surgical sutures. These tests were also performed in accordance with Good Laboratory Practice (GLP) regulations.

The LLNA test evaluated the skin sensitization potential of denatured albumin by administering an extract of denatured albumin to the skin of mice and measuring the proliferation of cells in lymph nodes draining the exposure site.

Representative portions of the denatured albumin strips were cut into pieces, placed into test tubes and prepared at a ratio of 60 cm² to 20 ml of extraction vehicle. Two different extract vehicles were used: 0.9% normal saline (NS) and dimethylsulfoxide (DMSO). Three doses of extract were prepared for each extract vehicle represented by three different denatured albumin sample surface areas per ml extract volume. The denatured albumin samples were extracted at 37° C. for 72 hours. The albumin extracts were then cooled, shaken, and decanted into sterile, dry glass vessels. Saline extracts were mixed with the detergent Pluronic L-92 to facilitate dose delivery. The extract was used within 24 hours of preparation.

Swiss mice (8 to 14 weeks old) were randomized and placed into groups of five animals each. Five mice per group were administered a 25 μl dose of albumin extract applied to the dorsum of each ear daily for three days. Five negative control mice received the same volume of vehicle administered in the same way, and five positive control mice received a known sensitizer (either 20% 2,4-dinitrobenzenesulfonic acid in NS or 0.5% dinitrochlorobenzene in DMSO). Each animal was observed daily for general health and clinical signs of toxicity according to a standard survival check paradigm. Animal weights were recorded on day 0 and day 5. Particular attention was paid to gross evidence of irritation or inflammation.

On the fifth day following dosing, each animal was injected with approximately 20 μCi of radiolabelled methylthymidine ([3H]TdR) via a tail vein injection. This isotope is rapidly incorporated into mitotically active cells (dividing lymphocytes). The isotope injection was monitored by inclusion of 0.1% Evans Blue dye for verification of delivery. The auricular lymph modes were then dissected bilaterally, isolated lymph node cells prepared, and radioactivity incorporation measured. The lymph nodes from each mouse were pooled but individual animal data were collected. Radioactivity in the lymph nodes harvested was measured and a Stimulation Index (SI) was calculated (SI=average radioactivity of albumin/average radioactivity of control). A SI greater than 3.0 indicates that the test material may be a sensitizer.

TABLE 1
Radioisotope Uptake and Stimulation Index with Saline
Extraction

| | [3H]-TdR Uptake | |
|---|---|---|
| Treatment | (DPM) | Stimulation Index |
| Negative control | 205.3 ± 94.7 | 1.0 |
| Positive control | 2706.6 ± 2613.9 | 13.18 |
| Test article extract | 300.2 ± 94.7 | 1.46 |

TABLE 2
Radioisotope Uptake and Stimulation Index with DMSO
Extraction

| | [3H]-TdR Uptake | |
|---|---|---|
| Treatment | (DPM) | Stimulation Index |
| Negative control | 497.0 ± 137.6 | 1.0 |
| Positive control | 9438.6 ± 7743.3 | 18.99 |
| Test article extract | 283.9 ± 76.7 | 0.57 |

Results of the LLNA testing showed that the denatured albumin extracts had a Stimulation Index significantly less than 3.0. Furthermore, the level of cell proliferation stimulated by the albumin extract was equivalent to the level of cell proliferation stimulated by the negative controls (Tables 1 and 2). These data demonstrated that the denatured albumin material did not produce skin sensitization and thus was biocompatible.

The intracutaneous reactivity test evaluated the skin irritation potential of denatured albumin by administering a saline and cottonseed oil extract of denatured albumin intracutaneously in rabbits and comparing the level of irritation produced locally with concurrent injections of the vehicle controls (i.e., normal saline and conttonseed oil).

More specifically, the denatured albumin material was cut into pieces, placed in test tubes, and prepared at a ratio of 60 cm² to 20 ml of extraction vehicle. Two different extract vehicles were used: 0.9% normal saline (NS) and cottonseed oil (CSO). The denatured albumin extracts and control vehicles were extracted for 72 hours at 37° C. The extracts were cooled, shaken, and decanted into a sterile, dry glass vessel. The extracts were used within 24 hours of preparation.

New Zealand White rabbits (female, >2.0 kg body weight) were randomized and housed individually. Each animal was weighed before testing and clipped on both sides of the spinal column to expose a sufficient test area for injection. Two denatured albumin extracts and two vehicle controls were injected into two rabbits.

Each rabbit received five sequential 0.2 ml intracutaneous injections of the albumin extract on the right side of the vertebral column and similar injections of the control vehicle on the left side. The second set of albumin extract and control vehicle injections were parallel and distal to the first injection sites. The animals were observed daily for abnormal clinical signs. The appearance of each injection site was noted at 24, 48 and 72 hours post injection.

The tissue reactions were rated for evidence of erythema and edema. The skin was lightly swabbed with dilute alcohol to enhance the appearance of any such reactions. The intradermal injection of CSO frequently elicits an inflammatory response. CSO scores greater than 2 are thus considered normal. Reactions were scored on a scale of 0 to 4 (0=no reaction; 1=slight reaction; 2=well-defined erythema/slight edema; 3=moderate to severe erythemal moderate edema; 4=severe erythema/severe edema) for both edema and erythema (2 scores per sites per time point). The scores for each albumin sample and control vehicle were determined and collected. Each score was divided by 12 (2 animals×3 observation periods×2 scoring categories) to determine the overall mean score for each test extract versus the corresponding control.

TABLE 3
Dermal Observations for Extraction with Normal Saline

| Rabbit | Control Scores | Test Extract Scores |
|---|---|---|
| #3654 | | |
| 24 hours | 0 | 0 |
| 48 hours | 0 | 0 |
| 72 hours | 0 | 0 |
| TOTAL | 0/6 | 0/6 |
| #3650 | | |
| 24 hours | 0 | 0 |
| 48 hours | 0 | 0 |
| 72 hours | 0 | 0 |
| TOTAL | 0/6 | 0/6 |
| COMPARATIVE RESULTS | 0 | |

TABLE 4
Dermal Observations for Extraction with Cottonseed Oil

| Rabbit | Control Scores | Test Extract Scores |
|---|---|---|
| #3654 | | |
| 24 hours | 5 | 5 |
| 48 hours | 1 | 5 |
| 72 hours | 1 | 2 |
| TOTAL | 7/6 | 12/6 |
| #3650 | | |
| 24 hours | 0 | 2 |
| 48 hours | 0 | 0 |
| 72 hours | 0 | 0 |
| TOTAL | 0/6 | 2/6 |
| COMPARATIVE RESULTS | 1.17 − 0.58 = 0.59^{A} | |

^{A}The value here is calculated by first dividing the total scores for each of the two rabbits by the number of observations (7/12 and 14/12)

The irritation reaction of the albumin extracts were compared to the vehicle controls and recorded over a 72-hour period according to the standard Irritation Scoring System. According to accepted test criteria, if the difference between the average scores for the extract of the test material and the vehicle control is less than or equal to 1.0, the test material is considered non-irritating. Results of the Intracutaneous Reactivity Test, shown in Tables 3 and 4, demonstrated that the denatured albumin extract was a non-irritant and thus would be considered biocompatible.

The bio-absorption of the denatured albumin material was assessed by analyzing the histology of laceration and resection sites treated with the denatured albumin material over a four week period. Seventy-three sections, from chronic laceration or resection sites from the liver of 11 pigs treated with the denatured albumin material, were evaluated by a veterinarian pathologist. The laceration and resection repair of sites was assessed in two pigs at the end of 2 weeks. The laceration and resection repair in the other nine pigs was evaluated at the end of 4 weeks. All of the laceration and resection sites showed complete healing characterized by fibrous scar (laceration sites) or fibrous capsule (amputation or resection sites). There was a moderate amount of residual albumin within the fibrous scar that showed on-going bioresorption. Thus, the denatured albumin sutures are slowly absorbed and allow the wounds closed with the sutures to heal.

Considered together, the in vitro and in vivo data verify that the denatured human serum albumin product to be used as surgical suture material is biocompatible and bio-absorbable. Therefore, the sutures of the present invention could be considered safe for use in animals, including humans. Furthermore, the sutures of the present invention meet the strength and flexibility standards of the United States Food and Drug Administration and the Department of Health and Aging of the Australian government.

Therefore, the present invention is a strong, flexible, biocompatible and bio-absorbable surgical filament (monofilament, psuedo-monofilament twisted, or multi-filament braided) suture formed from a denatured human albumin. In one embodiment, the denatured human albumin is present in the suture at a concentration of at least 47% w/v human serum albumin. In a preferred embodiment, the denatured human albumin suture material has a denatured albumin concentration of at least 50%.

In one embodiment, the biocompatible surgical suture is formed by molding liquid human albumin in a tubular mold or dye and denaturing said liquid human albumin by application of wet or dry heat at a temperature of from 85° C. to about 120° C. for 15 seconds to about 30 minutes at a pressure of from about 1 atmosphere to about 3 atmospheres.

In another embodiment, the biocompatible surgical suture is formed by cutting fine ribbons from denatured human albumin sheets. One of skill in the art will appreciate that other methods of manufacture of surgical sutures may be used and employed with the denatured albumin material of the present invention. Further, the surgical sutures of the present invention will be used by one of skill in a variety of applications depending on the situation encountered and will include but not be limited to wound repair and closure, either internally or externally in a human patient.

It should be appreciated by those skilled in the art that the conception and the specific embodiment disclosed might be readily utilized as a basis for modifying or redesigning the structures for carrying out the same purposes as the invention. It should be realized by those skilled in the art that such equivalent constructions do not depart from the spirit and scope of the invention as set forth in the appended claims.

What is claimed is:

1. A surgical suture comprising a denatured human albumin derived from denaturing a human serum albumin solution having an albumin concentration of from about 47% w/v to about 58% w/v.

2. The suture of claim 1 wherein the albumin concentration of the albumin solution ranges from about 50% w/v to 54% w/v.

3. The suture of claim 1, wherein the suture is sterilized with heat, gamma radiation, or ethylene oxide gas.

4. The suture of claim 1, wherein the suture has a yield strength of at least 400 kilopascals.

5. The suture of claim 4, wherein the yield strength of the suture ranges from about 800 kilopascals to about 1200 kilopascals.

6. The suture of claim 1, wherein the suture has a Young's modulus of elasticity of less than 4000 kilopascals.

7. The suture of claim 6, wherein the Young's modulus of elasticity ranges from about 2500 kilopascals to about 3500 kilopascals.

8. The suture of claim 1, wherein the suture is biocompatible and bioabsorbable.

9. A surgical suture consisting essentially of a denatured human albumin.

10. The suture of claim 9, wherein the suture has a denatured human albumin concentration of at least 47% w/v.

11. The suture of claim 10, wherein the albumin concentration is at least 50% w/v.

12. The suture of claim 9, wherein the suture is sterilized with heat, gamma radiation, or ethylene oxide gas.

13. The suture of claim 9, wherein the suture has a yield strength of at least 400 kilopascals.

14. The suture of claim 9, wherein the suture has a Young's modulus of elasticity of less than 4000 kilopascals.

15. A surgical suture containing a denatured human serum albumin at a concentration of at least 50% w/v, wherein the suture has a yield strength ranging from about 800 kilopascals to about 1200 kilopascals and a Young's modulus of elasticity ranging from about 2500 kilopascals to about 3500 kilopascals.

16. A method for making a suture material comprising the steps of:
obtaining an albumin solution having a concentration of human serum albumin ranging from about 47% w/v to about 58% w/v;
casting the albumin solution into a predetermined shape; and
denaturing the human serum albumin by heating the albumin to at least 85° C. at a pressure of at least 1 atmosphere for at least 15 seconds.

17. The method of claim 16, wherein the albumin solution contains about 50% w/v to about 54% w/v of human serum albumin.

18. The method of claim 16, wherein the albumin is cast into a denatured albumin lamina between two aligned nonporous sheets.

19. The method of claim 18, wherein the denatured albumin lamina is cut to form the suture material.

20. The method of claim 16, wherein the albumin is cast by injecting the albumin solution into a tubular mold or dye.

21. The method of claim 16, wherein the human serum albumin is denatured by heating the albumin solution from 15 seconds to 30 minutes between 85° C. and 120° C. at a pressure ranging from about 1 atmosphere to about 3 atmospheres.

22. The method of claim 16, wherein the human serum albumin is denatured by autoclaving the cast albumin solution.

24. A method for making a biocompatible suture material by molding an albumin solution containing 47% w/v to 58% w/v of human serum albumin solution in a tubular mold and heating the molded albumin solution at a temperature ranging from 85° C. to 120° C. for about 15 seconds to about 30 minutes at a pressure ranging from about 1 atmosphere to about 3 atmospheres.

25. The method of claim 24, wherein the suture material is sterilized with heat, gamma radiation, or ethylene oxide gas.
